(19) United States
(12) Patent Application Publication
(10) Pub. No.: US 2014/0000614 A1
(43) Pub. Date: Jan. 2, 2014

(54) Nasal Mask

(71) Applicant: Eric CHANG, Taichung City (TW)

(72) Inventor: Eric CHANG, Taichung City (TW)

(21) Appl. No.: 13/539,828

(22) Filed: Jul. 2, 2012

(51) Int. Cl. A61M 16/06

(52) U.S. Cl. 128/205.25

(57) ABSTRACT

A nasal mask includes a main body having an annular inner rim and an annular outer rim, two connection members respectively connected to two opposite sides of the main body, and two engaging holes each formed in proximity to a junction between one connect ion member and the main body. The inner rim is formed with an annular shoulder and a seal ring that projects from the shoulder. A head strap unit has two opposite ends connected to the connect ion members. A nose unit includes a main plate having a peripheral edge abutting against the shoulder and the seal ring, two nasal prong members connected to the main plate, and two engaging plates to engage the engaging holes. An air supply unit is connected to the outer rim.

### BACKGROUND OF THE INVENTION

1. Field of the Invention

The invention relates to a medical device, and more particularly to a nasal mask.

2. Description of the Related Art

Referring to FIGS. 1 and 2, a conventional nasal mask is shown to include a main body 1, a head strap 2 connected to two opposite sides of the main body 1, a nose unit 3 connected to an inner side of the main body 1, and an air supply tube 4 connected to the main body 1 opposite to the nose unit 3. The main body 1 is made in one piece from silicone, and has an inner wall 101, and an outer wall 102 opposite to the inner wall 101. The inner wall 101 defines an engaging hole 103 to receive engagingly the nose unit 3. The outer wall 102 defines a connecting hole 104. The air supply tube 4 is connected to and is positioned in the connecting hole 104.

When the conventional nasal mask is worn by a user and an external force is acted on the head strap 2, the main body 1 is adversely affected such that the nose unit 3 may be disengaged from the main body 1, and air may leak from the engaging hole 103 to result in insufficient supply of air, thereby influencing the sleep quality of the user.

### SUMMARY OF THE INVENTION

Therefore, an object of the present invention is to provide a nasal mask that is capable of overcoming the aforesaid drawbacks of the prior art.

According to the present invention, a nasal mask comprises a main body unit, a head strap unit, a nose unit, and an air supply unit. The main body unit includes a main body defining a chamber, two connection members respectively connected to two opposite sides of the main body, and two engaging holes each formed in proximity to a junction between one of the connection members and the main body. The main body has an inner end that is adapted to contact a user's face and that has an annular inner rim defining an inner opening communicating spatially with the chamber, and an outer end that is opposite to the inner end and that has an annular outer rim defining a connecting hole communicating spatially with the chamber. The annular inner rim is recessed steppedly to form an annular recess that extends around the inner opening, and an annular shoulder that is formed as an annular floor for the annular recess and that faces in a direction toward the user's face. The annular inner rim is formed with a seal ring that projects from the annular shoulder. The head strap unit has two opposite ends connected respectively to the connection members. The nose unit includes a main plate having a peripheral edge received engagingly in the annular recess and abutting against the annular shoulder and the seal ring, two nasal prong members connected to the main plate, and two engaging plates projecting respectively from two opposite sides of the peripheral edge to engage the engaging holes, respectively.

The air supply unit is opposite to the nose unit, and is connected to the annular outer rim.

### BRIEF DESCRIPTION OF THE DRAWINGS

Other features and advantages of the present invention will become apparent in the following detailed description of the preferred embodiment with reference to the accompanying drawings, of which:

FIG. 1 is a partly sectional view of a conventional nasal mask;

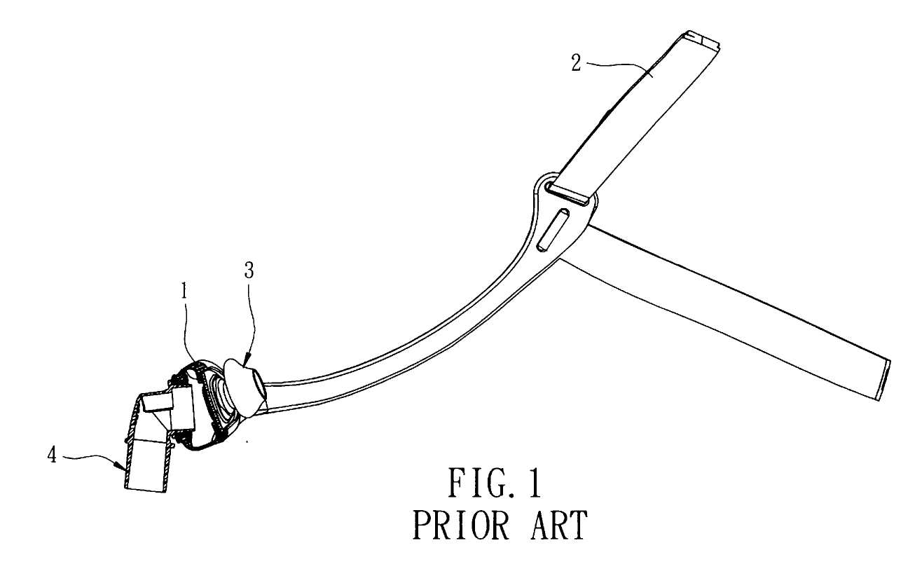

FIG. 2 is a an enlarged fragmentary partly sectional view of the conventional nasal mask;

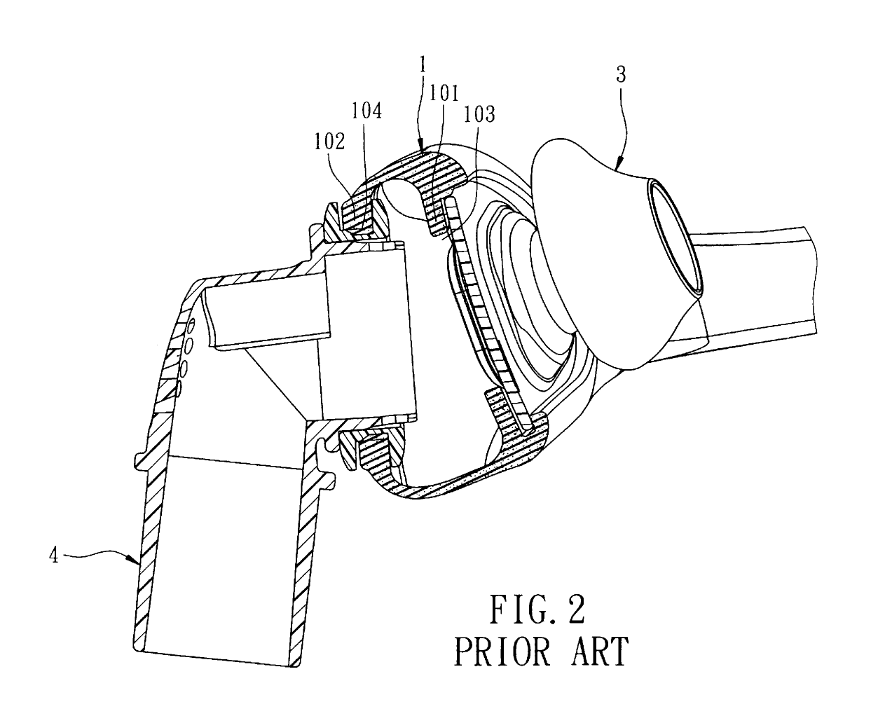

FIG. 3 is a perspective view of a nasal mask according to the preferred embodiment of the present invention;

FIG. 4 is an enlarged exploded perspective view of the preferred embodiment;

FIG. 5 is an enlarged partly sectional view of the preferred embodiment;

FIG. 6 is a sectional view of the preferred embodiment taken along line VI-VI of FIG. 5;

FIG. 7 is an enlarged fragmentary partly exploded sectional view of the preferred embodiment; and

FIG. 8 is a view similar to FIG. 7, but with a nose unit of the preferred embodiment assembled on a main body of the nasal mask.

### DETAILED DESCRIPTION OF THE PREFERRED EMBODIMENT

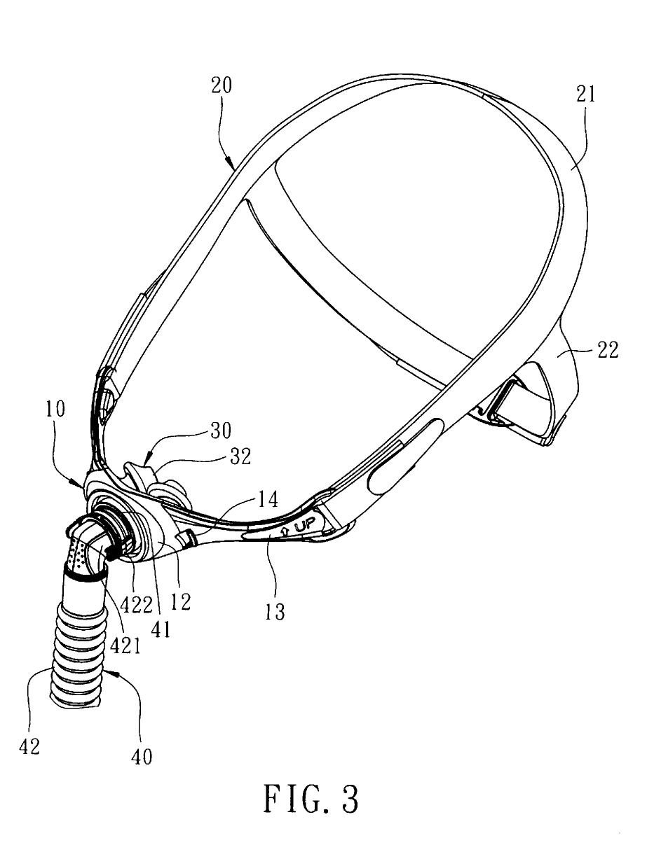

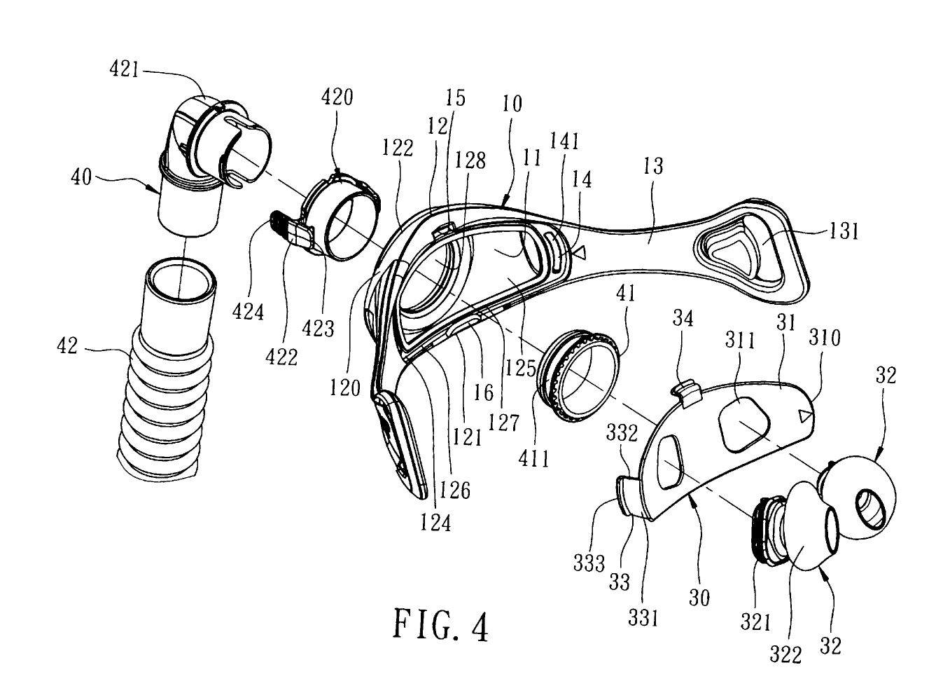

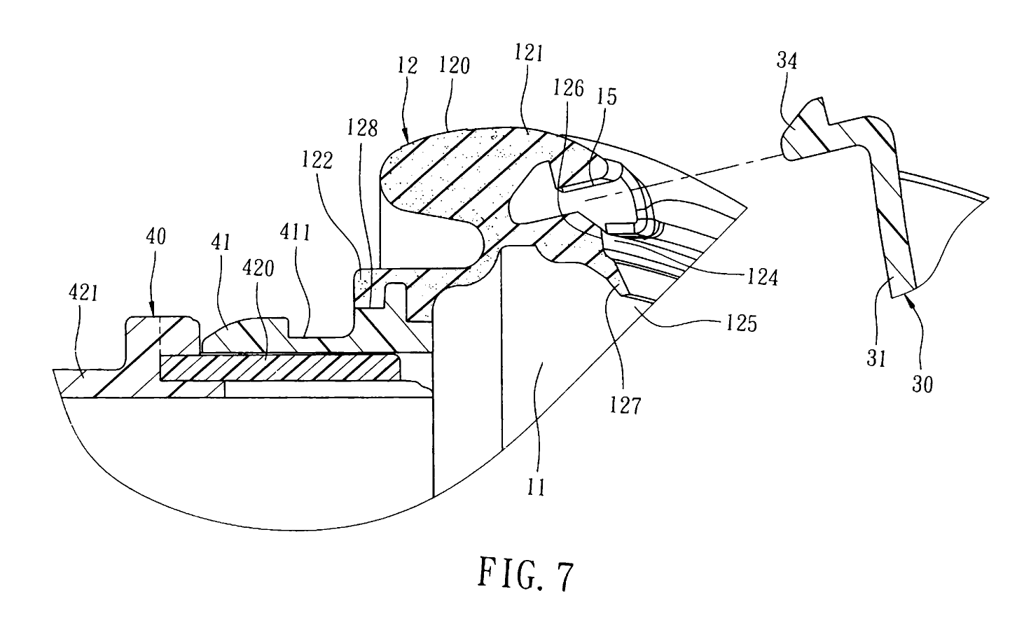

Referring to FIGS. 3 to 8, a nasal mask according to the preferred embodiment of this invention is shown to comprise a main body unit 10, a head strap unit 20, a nose unit 30, and an air supply unit 40.

The main body unit 10 is made of silicone, and is formed of a unitary construction. The main body unit 10 includes a main body 12 defining a chamber 11, two connection members 13 respectively connected to two opposite sides of the main body 12, two engaging holes 14 each formed in proximity to a junction between one of the connection members 13 and the main body 12, a locking hole 15 provided in the main body 12, and a stop flange 16 aligned with the locking hole 15 in a top-bottom direction.

The main body 12 has an inner end that is adapted to contact a user's face and that has an annular inner rim 121, an outer end that is opposite to the inner end and that has an annular outer rim 122, and a chamber wall 120 that interconnects the inner and outer rims 121, 122. The annular inner rim 121 defines an inner opening 125 communicating spatially with the chamber 11, and is recessed steppedly to form an annular recess 124 that extends around the inner opening 125, and an annular shoulder 126 that is formed as an annular floor for the annular recess 124 and that faces in a direction toward the user's face. Further, the annular inner rim 121 is formed with a seal ring 127 that projects from the annular shoulder 126 towards the inner opening 125. The annular outer rim 122 defines a connecting hole 128 communicating spatially with the chamber 11.

Each of the connection members 13 has a through hole 131 formed in a free end thereof.

Each of the engaging holes 14 has an entrance end 141 extending through the shoulder 126, and an exit end 142 extending through the chamber wall 120 opposite to the entrance end 141.

The locking hole 15 is formed in the annular inner rim 121 above the inner opening 125, and is configured as a blind hole.

The stop flange 16 projects from the annular inner rim 121 toward the inner opening 125.

The head strap unit 20 includes a first head strap 21 having two opposite ends connected respectively to the through holes 131 in the connection members 13, and a second head strap 22 having two opposite ends connected to the first head strap 21, as shown in FIG. 3.

The nose unit 30 includes a main plate 31 having a peripheral edge 310 received engagingly in the annular recess 124 and abutting against the annular shoulder 126 and the seal ring 127, two nasal prong members 32 connected to the main plate 31, two engaging plates 33 projecting respectively from two opposite sides of the peripheral edge 310 to engage the engaging holes 14, respectively, and a locking plate 34 connected to the peripheral edge 310 between the engaging plates 33.

In this embodiment, the main plate 31 is formed with two prong holes 311 communicating with the chamber 11. The nasal prong members 32 are hollow and are made of silicone. Each of the nasal prong members 32 has an insert tube 321 inserted into a respective prong hole 311, and a nasal insert 322 connected to the insert tube 321 for insertion into a respective nostril of the user. Each of the engaging plates 33 has a connecting end 331 connected to the peripheral edge 310 of the main plate 31 and extending into the entrance end 141, an engaging end 332 extending outwardly of the exit end 142, and an engaging hook 333 formed on the engaging end 332. The engaging hook 333 is engaged with an outer surface of the chamber wall 120 in proximity to the exit end 142. The locking plate 34 has a barb-like structure, and is press-fitted into the locking hole 15. When the main plate 31 is inserted into the annular recess 124 via the inner opening 125, the stop flange 16 abuts against an inner surface of the main plate 31.

The air supply unit 40 is opposite to the nose unit 30, and is connected to the annular outer rim 122. The air supply unit 40 includes an annular connecting member 41 fittingly engaged in the connecting hole 128, and an air supply tube 42. The annular connecting member 41 has an outer surface formed with an annular groove 411. The air supply tube 42 has a head portion 421 inserted into the annular connecting member 41, a sleeve member 420 sleeved around the head portion 421, and two resilient securing hooks 422 connected respectively to two opposite sides of the sleeve member 420. Each of the securing hooks 422 has a hook portion 423 engaged to the annular groove 411, and a press portion 424 opposite to the hook portion 423. Through the biasing characteristics of the resilient securing hooks 422, engagement of the hook portions 423 of the securing hooks 422 with the annular groove 411 can be maintained. By pressing the pressing portions 424 of the securing hooks 422, the hook portions 423 can be disengaged from the annular groove 411.

To use the nasal mask of the present invention, the nasal inserts 322 of the nasal prong members 32 are inserted respectively into the nostrils of the user, after which the first and second head straps 21, 22 of head strap unit 20 are placed over the head of the user.

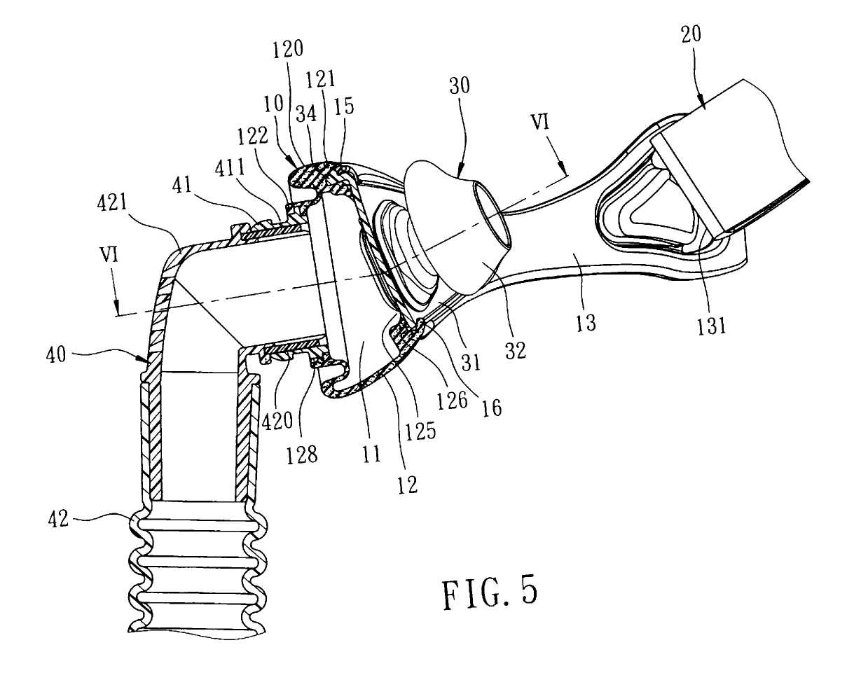

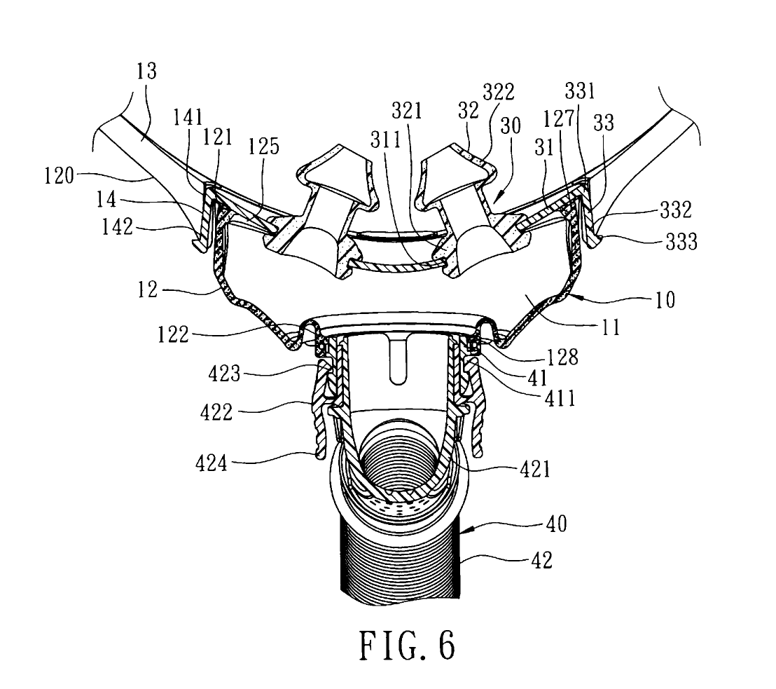

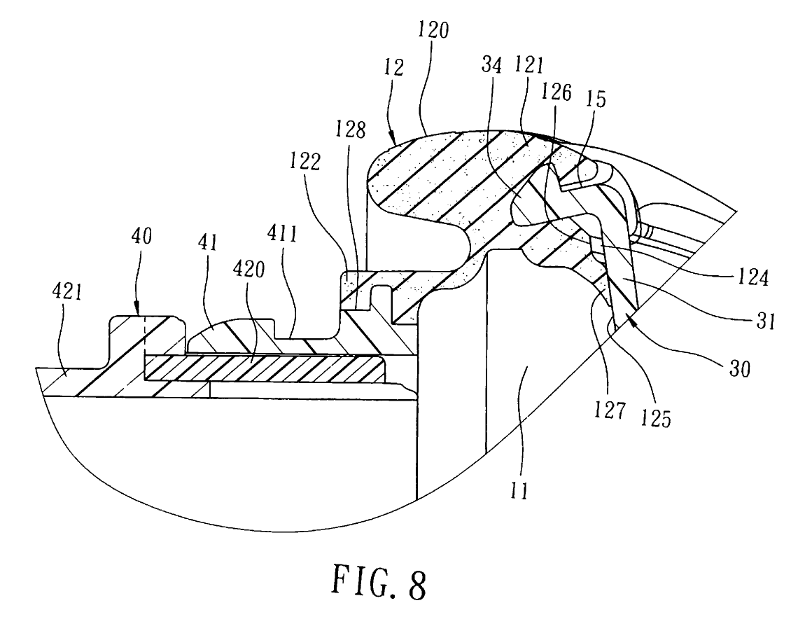

With reference to FIGS. 5 and 6, when a user wears the nasal mask of the present invention, air is supplied into the chamber 11 of the main body unit 10 through the air supply unit 40, and from the chamber 11, the air passes through the nasal prong members 32 and directly into the nostrils of the user for inhalation. At this state, the nose unit 30 is positioned through insertion of the two nasal prong members 32 into the user's nostrils, and a back pressure generated by the air in the chamber 11 forces the annular shoulder 126 to abut against the main plate 31, thereby pressing the seal ring 127 against the main plate 31 (see FIG. 8). Hence, an airtight seal exists between the nose unit 30 and the main body unit 10.

When an external force is acted on the nasal mask of the present invention during use thereof, the head strap unit 20, the connection members 13, or the main body unit 10 may be affected. However, because the engaging plates 33 are respectively engaged to the engaging holes 19, the engaging hooks 333 of the engaging plates 33 are engaged with the outer surface of the chamber wall 120, the locking plate 34 is engaged to the locking hole 15, and the stop flange 16 abuts against the inner surface of the main plate 31, the main plate 31 of the nose unit 30 can be stably positioned relative to the main body 12. That is, the nose unit 30 cannot be easily disengaged from the main body unit 10, thereby ensuring normal supply of air to the user. Furthermore, an airtight seal is effected through abutment of the seal ring 127 against the main plate 31.

When the air supply unit 40 is acted on by an external force during use of the nasal mask of the present invention, the annular outer rim 122 of the main body 12 may be affected. Similarly, because the engaging plates 33 are respectively engaged to the engaging holes 14, the engaging hooks 333 of the engaging plates 33 are engaged with the outer surface of the chamber wall 120, the locking plate 34 is engaged to the locking hole 15, and the stop flange 16 abuts against the inner surface of the main plate 31, the nose unit 30 can be stably positioned relative to the main body 12, and cannot be easily disengaged from the main body unit 10. Hence, normal supply of air to the user can be ensured.

Moreover, because the main body unit 10 is made in one piece from silicone, the engaging plates 33 can be quickly engaged to or disengaged from the respective engaging holes 14, so that assembly and disassembly thereof is relatively easy, and the main body unit 10 cannot be easily damaged. In addition, through the configuration of the securing hooks 422 of the air supply unit 40, the air supply tube 42 can be easily assembled to and disassembled from the annular connecting member 41.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

What is claimed is:

1. A nasal mask comprising:
a main body unit including a main body defining a chamber, two connection members respectively connected to two opposite sides of said main body, and two engaging holes each formed in proximity to a junction between one of said connection members and said main body, said main body having an inner end that is adapted to contact a user's face and that has an annular inner rim defining an inner opening communicating spatially with said chamber, and an outer end that is opposite to said inner end and that has an annular outer rim defining a connecting hole communicating spatially with said chamber, said annular inner rim being recessed steppedly to form an annular recess that extends around said inner opening, and an annular shoulder that is formed as an annular floor for said annular recess and that faces in a direction toward the user's face, said annular inner rim being formed with a seal ring that projects from said annular shoulder;
a head strap unit having two opposite ends connected respectively to said connection members;
a nose unit including a main plate having a peripheral edge received engagingly in said annular recess and abutting against said annular shoulder and said seal ring, two nasal prong members connected to said main plate, and two engaging plates projecting respectively from two opposite sides of said peripheral edge to engage said engaging holes, respectively; and
an air supply unit opposite to said nose unit and connected to said annular outer rim.

2. The nasal mask as claimed in claim 1, wherein:
said main body has a chamber wall that interconnects said inner and outer rims;
each of said two engaging holes in said main body unit has an entrance end extending through said shoulder, and an exit end extending through said chamber wall opposite to said entrance end; and
each of said two engaging plates of said nose unit has a connecting end connected to said peripheral edge of said main plate and extending into said entrance end, and an engaging end extending outwardly of said exit end of said main body unit.

3. The nasal mask as claimed in claim 2, wherein each of said engaging plates of said nose unit has an engaging hook formed on said engaging end, and said engaging hook is engaged with an outer surface of said chamber wall in proximity to said exit end.

4. The nasal mask as claimed in claim 3, wherein said main body further includes a locking hole formed in said annular inner rim above said inner opening, and said nose unit further includes a locking plate connected to said peripheral edge between said engaging plates and engaged to said locking hole.

5. The nasal mask as claimed in claim 4, wherein said main body unit further includes a stop flange projecting toward said inner opening from said annular inner rim to abut against an inner surface of said main plate.

6. The nasal mask as claimed in claim 1, wherein said air supply unit includes an annular connecting member fittingly engaged in said connecting hole in said main body unit, and an air supply tube, said annular connecting member having an outer surface formed with an annular groove, said air supply tube having a head portion inserted into said annular connecting member, and two securing hooks connected to two opposite sides of said head portion, each of said securing hooks having a hook portion engaging said annular groove, and a press portion opposite to said hook portion.
